(19) United States
(12) Patent Application Publication
(10) Pub. No.: US 2025/0169776 A1
(43) Pub. Date: May 29, 2025

(54) A RADIATION PROTECTION SHIELDING SYSTEM AND A METHOD FOR COVERING A PATIENT

(71) Applicant: TEXRAY AB, Gothenburg (SE)

(72) Inventors: Fredrik GELLERSTEDT, Gothenburg (SE); Petra APELL, Onsala (SE); Oskar ANGERÅS, Gothenburg (SE); Truls RAMUNDDAL, Gothenburg (SE)

(73) Assignee: TEXRAY AB, Gothenburg (SE)

(21) Appl. No.: 18/840,173

(22) PCT Filed: Feb. 24, 2023
(86) PCT No.: PCT/SE2023/050165

§ 371 Date: Aug. 21, 2024

(30) Foreign Application Priority Data
Feb. 25, 2022 (SE) 2250265-2

(51) Int. Cl. A61B 6/10; A61B 6/04; A61B 46/20

(52) U.S. Cl. CPC A61B 6/107; A61B 6/0407; A61B 46/20

(57) ABSTRACT

A radiation protection shielding system is disclosed, which comprises at least one radiation protection drape sized to at least partially cover the patient from a proximity of a radiated area of the patient to the feet of the patient. The radiation protection drape comprises at least a first section a second section. The first section is partitioned from the second section from a first edge of the radiation protection drape towards a second edge of the radiation protection drape.

### FIELD OF THE INVENTION

This invention pertains in general to the field of radiation protection devices for protection against ionizing radiation, particular for use in medical interventions. More particularly, the invention relates to a radiation protection shielding system comprising at least one radiation protection drape sized to at least partially cover the patient from a proximity of a radiated area of the patient to the feet of the patient. The radiation protection drape may comprise at least a first section and a second section. The first section may be partitioned from the second section from a first edge of the radiation protection drape towards a second edge of the radiation protection drape.

### BACKGROUND OF THE INVENTION

In interventional procedures with radiologic guidance using radiation systems, such as X-ray radiation systems, the clinical staff, such as the endovascular clinician, is continuously exposed to ionizing radiation. The vast majority of the ionizing radiation the clinical staff is exposed to is scattered radiation from the patient and operating table.

The clinical staff protects themselves from radiation mainly in two ways, here called the conventional systems; 1) radiation shielding devices (exemplified in roof or floor mounted protective shields, radiation protection shields mounted under the operating table; and 2) personal radiation shielding garments (such as lead or no-lead aprons, thyroid protection and lead eyeglasses). Alternative radiation protection systems may be radiation protection patches that are applied on the patient as to create zones with lower scatter radiation in the clinical environment. Another example is a system that dresses the operator with a floor or roof mounted radiation protection garment, thus, to maximize radiation protection without the operator needing to carry the weight of a personal radiation shielding garment. Still another example is to reduce radiation by a system of protective panels around the interventional area. However, there is no optimal radiation protection solution as conventional as well as the alternative radiation protective systems reveal flaws in protection to the staff. In the conventional radiation garment systems, typically only 75% of the body of the staff is protected, leaving arms, legs and head exposed to high doses of radiation. In the example with the system that dresses the operator with a floor or roof mounted radiation protection garment (e.g. Zero-Gravity, by TIDI Products, USA), the system only allows one person in the operating room to be protected, the other staff will remain dependent on conventional radiation protection equipment.

The conventional protection system used today is accepted as the standard and is mandatory in all interventional procedures with radiologic guidance using radiation systems, such as endovascular procedures, to reduce the radiation dose to the clinical staff. Every member of the staff has a dosimeter at their left breast pocket under their lead aprons for monitoring of personal radiation exposure. For example, in endovascular operation rooms with modern dose optimized x-ray systems equipped with a protection system, such as described above, the yearly cumulative doses measured under the lead aprons on catheterization laboratory staff are in the range of 2-4% of the yearly allowed dose of 20 mSv. Although occupational radiation dose limits are being monitored and seldom reached, chronic exposure to low dose radiation have been shown to have harmful biological effects that may become apparent years later, such as cancer and cataract. Further, as the dose is measured on the torso, under the radiation protection garment and any accumulated dose on arms, legs and head is not considered in the annual dose limit.

The personal lead or no-lead protection aprons weigh approximately 4-8 kg, pending on size and protection level. The weight of the lead aprons put significant strain on the clinician that needs to carry these when working inside the operating room. Orthopedic injuries and disorders to the back, neck, shoulders, hips and knees as well as chronic fatigue are common for workers in the interventional operating environment as a consequence of carrying heavy personal lead protection.

Hence, an effective radiation protection system to protect against radiation exposure to the staff in interventional procedures with radiologic guidance and at the same time to reduce the risk of orthopedic injury and disorders among the staff would be advantageous.

U.S. Pat. No. 4,670,658 discloses a multi-ply sheet having a center ply support matrix in which the radio opaque compound barium sulfate is distributed as a powder and is supported by the matrix. The barium sulfate is present in an amount sufficient to block scatter radiation produced by the medical radiology procedures. The multi-ply sheet is a single sheet that is disposable and designed with cost in mind. The radiation protective material suggested is barium sulphate, which is known as a cost efficient material but has at the same time a relatively low protective performance to radiation shielding. Conformity over the patient and operating table is critical to avoid leaks of radiation and properly reduce the radiation exposure to the clinical staff and although the suggested solutions in U.S. Pat. No. 4,670,658 may reduce radiation exposure, there is an evident risk that the inherent constitution of the solution may jeopardize the radiation barrier since the barium sulfate powder makes the multi-ply sheet stiff and brittle.

### SUMMARY OF THE INVENTION

Accordingly, embodiments of the present invention preferably seek to mitigate, alleviate or eliminate one or more deficiencies, disadvantages or issues in the art, such as the above-identified, singly or in any combination by providing embodiments of a radiation protection system according to embodiments of the invention.

According to a first aspect of the invention, a radiation protection shielding system comprises at least one radiation protection drape sized to at least partially cover the patient from a proximity of a radiated area of the patient to the feet of the patient. The radiation protection drape comprises at least a first section and a second section. The first section may be partitioned from the second section from a first edge of the radiation protection drape towards a second edge of the radiation protection drape.

The first section may be partitioned from the second section from the first edge to the second edge of the radiation protection drape and being separable from the second section. Alternatively or additionally, the first section is partitioned from the second section from the first edge partly to the second edge. The first section may be partly separated from the second section.

The first section may have a varying radiation protection level across the first section.

The second section may comprise a first portion and a second portion. The first portion and the second portion may be separated along an edge of each of the first portion and the second portion. Furthermore, each of the first portion and the second portion may comprise a fastener to temporarily fasten and unfasten the first portion to/from the second portion.

The radiation protection drape and/or garment may comprise one or several radiation attenuating layers positioned in between two protecting layers of non-radiation attenuating material attached to each other in a perimeter, hence separating the metals in the attenuating material from the outside of the radiation protection drape and/or garment.

The radiation protection drape may comprise at least one fenestration for a femoralis area of the patient. The fenestration may be located along an edge of the first section and an edge of the second section, along which the first section and the second section are partitioned.

The second edge of the radiation protection drape may be located at the fenestration. The first section and the second section may be partitioned from the first edge to the second edge of the radiation protection drape.

The radiation protection drape may comprise a third section separated from the first section and the second section. The third section may be sized to partially cover the fenestrated area of the radiation protection drape.

The radiation protection drape may comprise at least one fastening device located at where the first section is partitioned from the second section. The fastening device may, e.g., comprise hooks and loops, and/or magnets.

The radiation protection drape may be sized to extend over the rim of an operating table when positioned over a patient lying on the operating table. Furthermore, the radiation protection drape may be sized to extend over the rim of the operating table and at least partially towards to the floor on at least one side of the operating table, most preferably at least 10 cm over the rim of the operating table. Hence, the radiation protection drape may cover the side of the patient when lying on the operating table.

The radiation protection shielding system may further comprise at least one floor or ceiling mounted radiation shielding screen to be positioned between clinical staff and a radiated area at an upper body portion of the patient.

The radiation protection shielding system may further comprise at least one floor or table mounted radiation protection drape section configured to cover at least a part of an area under the table, such as being sized to cover least one long side and/or one foot-end side of the operating table.

The radiation protection drape may have a radiation protection level corresponding to 0.25 to 1 mm lead equivalency, most preferably 0.35-0.7 mm lead equivalency.

The radiation protection shielding system may comprise a radiation protection garment. The radiation protection garment may comprise at least one panel sized to extend over at least the front side of a torso of a wearer. The panel may have a radiation protection level of 0.05 to 0.35 mm lead equivalency. Hence, the radiation protection drape may have a higher radiation protective level than the panel of the radiation protection panel. Furthermore, when made of the same material, the panel of the radiation protection garment may be lighter per surface area than the radiation protection drape. The staff may be protected from all or substantially all of the radiation scatter from the patient by the radiation protection drape, and from any residual radiation scatter by the radiation protection garment. Since the residual radiation scatter will be low, the radiation protection garment can be made lighter. Thus the embodiments of the invention improves working environment for the staff in the operating room, while at the same time reduce the risk for work related orthopedic injuries and disorders to the back, neck, shoulders, hips and knees as well as chronic fatigue as a consequence of carrying heavy personal lead protection. These benefits are provided while at the same time improving the radiation protection for the staff.

Other embodiments of the invention comprise a radiation protection drape having slits and/or openings in proximetry to the left and/or right radialis area of the patient. Such slit or opening allows for the patients arm to extend through the radiation barrier, thus to provide easy access to the radialis entry point, with little additional radiation exposure to the clinical staff.

In yet another embodiment of the invention, the radiation protection drape to be positioned on the patient comprises one attenuating compound whereas a radiation protection garment to be used by a clinical practitioner comprises another attenuating compound.

The radiation protection drape to be positioned on the patient may have a attenuating compound comprising primarily of Antimony. The radiation protection garment to be used by the clinical practitioner may comprise an attenuating compound primarily consisting of Bismuth and/or Wolfram. With different attenuating compounds in the radiation protection drape and the radiation protection garment, the protection can be made more efficient. Each attenuating compound can be selected to shield against radiation with a specific wavelength and sequenced in this order thus to more efficiently reduce radiation. Therefore, the radiation protection per surface area is more efficient, and the drape and garment can be made lighter with the benefits described herein.

In another embodiment of the invention, the radiation protection garment comprises one or several textile radiation protection panels integrated in a set of pants and shirt thus to construct a scrub suit, such panels essentially covering the front and/or the back of the wearer. The embodiments of the invention provide numerous benefits for clinical staff and patient. For example, the invention provides for enhanced protection for the clinical staff including protection for the head, arms and legs without the need for a heavy radiation protection garment. Any additional protection garment, such as a lower protection garment in order to protect from residual or stray scatter radiation, can have reduced protective level and therefore be made lighter. The removal of weight from the clinical staff will benefit in stamina and avoiding work related injuries and disorders, such as described above. It will further also improve the movability and range of the operator and staff. The inherent weight of the patient drape has been commented as calming and caressing to the patient in the same functionality as having a weight blanket. Furthermore, the benefits described above are available while the safety of the patient is not compromised due to any inaccessibility to body parts of the patient in emergency situations. Further benefits and/or advantages of embodiments of the invention are described in the detailed description of the invention.

It should be emphasized that the term “comprises/comprising” when used in this specification is taken to specify the presence of stated features, integers, steps or components but does not preclude the presence or addition of one or more other features, integers, steps, components or groups thereof.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1a is a perspective view of embodiments of the invention;

FIG. 1 is a top view of embodiments of the invention in relation to a patient lying on an operating table;

FIG. 2 is a perspective view of the first section of the protection drape of embodiments of the invention relative an operating table;

FIGS. 3a-3b are perspective views of the second section of the protection drape of embodiments of the invention, in an unfolded and folded position, relative an operating table;

FIG. 4 is a perspective view of embodiments of the invention relative to an operating table;

FIG. 5-7 are tables with data from Example 1, Example 2, and Example 3, respectively; and

FIG. 8 is a top view of embodiments of the invention discussed relative to Example 4.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention.

The following description focuses on embodiments of the present invention applicable as a radiation protection system for medical procedures wherein radiologic guidance using radiation systems, such as X-ray radiation systems, continuously exposes the endovascular staff to ionizing radiation. However, it will be appreciated that the invention is not limited to this application but may be applied to many other procedures, such as other areas where a radiation protection system may be used to protect the staff from scatter radiation.

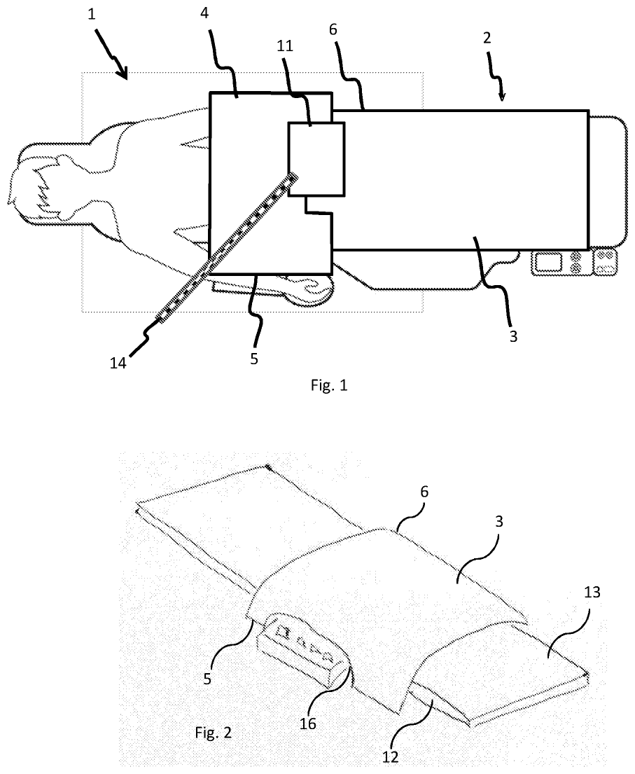

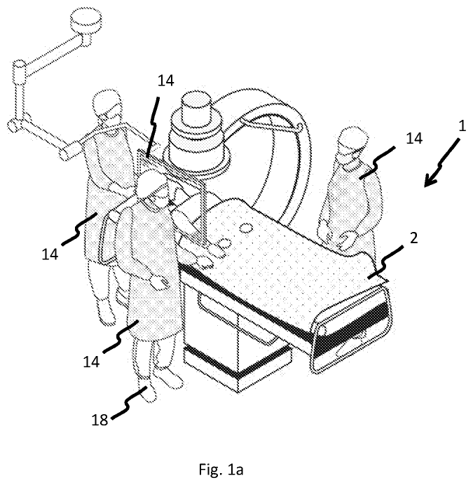

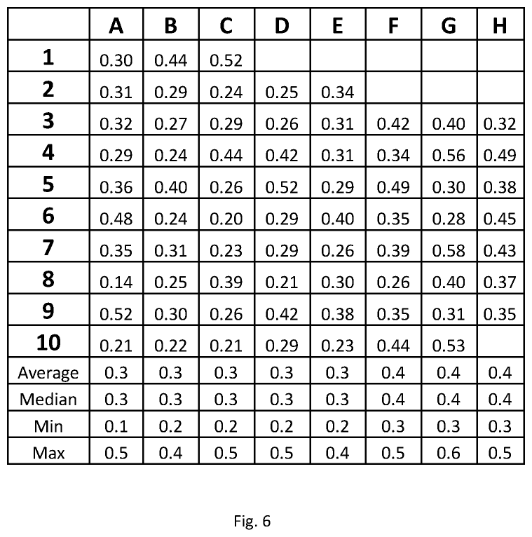

FIG. 1a illustrates embodiments of a radiation protection shielding system 1, which comprises at least one radiation protection drape 2 with at least one section. In some embodiments, the radiation protection shielding system also includes radiation protective garments 17 for staff in the operating room, which will be further disclosed below. In some embodiments, the radiation protection shielding system includes a radiation shielding screen 14 illustrated in FIG. 1a is also included in the radiation protective shielding system. Embodiments of the radiation protection shielding system can comprise the radiation protection drape 2 together with the radiation protection garment 17 and/or the radiation shielding screen 14. Embodiments can also comprise a radiation protection drape section 15, illustrated in FIG. 4, extending from the edge of the operating table towards the floor. The first operator 18 is illustrated standing on the long side of the operating table.

FIG. 1 illustrates embodiments of a radiation protection shielding system 1, which comprises at least one radiation protection drape 2. The radiation protection drape 2 comprises at least a first section 3 and a second section 4. The first section 3 may be partitioned from the second section 4 from a first edge 5 of the radiation protection drape 2 towards a second edge 6 of the radiation protection drape. Hence, the radiation protection drape 2 may be completely or partially partitioned, i.e. split or divided, into multiple sections.

The radiation protection drape 2 may be sized to at least partially cover the patient from a proximity of a radiated area of the patient to the feet of the patient. In some embodiments, the radiation protection drape 2 may be sized to cover the feet of the patient. The radiation protection drape 2 may be sized to cover the patient from the torso, as is illustrated in FIG. 1. The total length of the radiation protection drape 2 may be in the range of 60-150 cm, such as in the range of 80-110 cm. The total width of the radiation protection drape 2 may be in the range of 80-160 cm, such as in the range of 90-110 cm. The length of the first section 3 may be in the range of 20-130 cm, such as in the range of 30-60 cm. The total width of the first section 3 may be in the range of 80-160 cm, such as in the range of 90-110 cm. The length of the second section 4 may be in the range of 30-90 cm, such as in the range of 50-60 cm. The total width of the second section 4 may be in the range of 90-180 cm, such as in the range of 100-130 cm. Hence, the radiation protection drape 2 may be sized to cover the front and the sides of the patient when lying on the operating table.

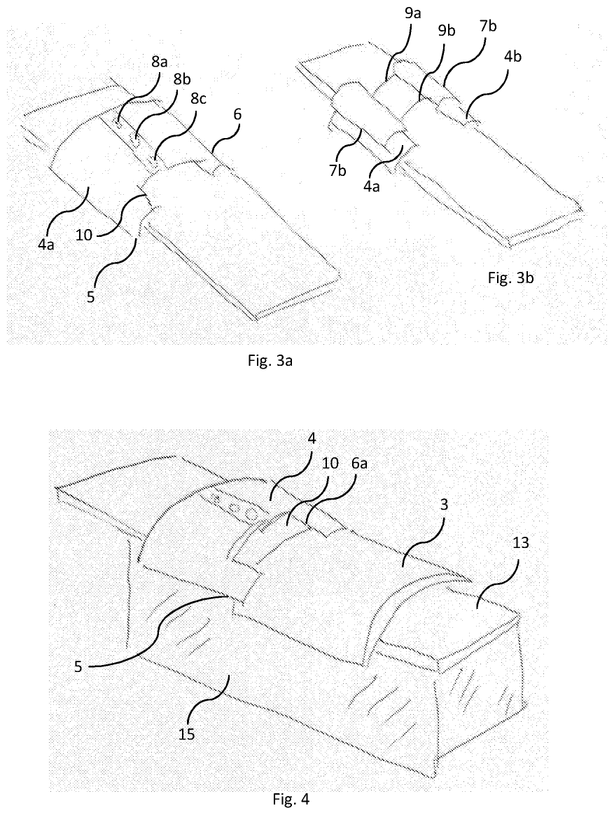

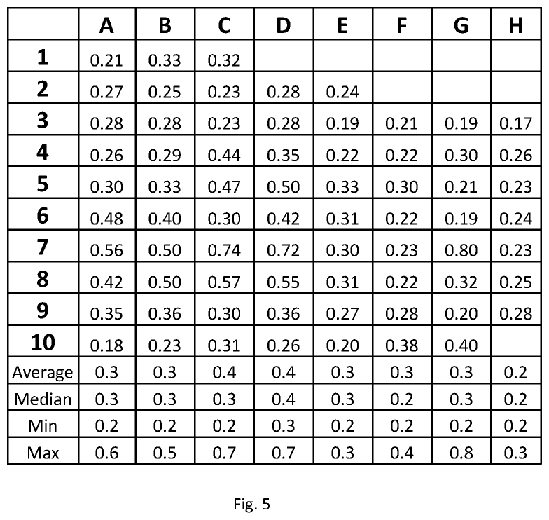

In some embodiments such as is illustrated in FIGS. 2 and 3a, the first section 3 is partitioned from the second section 4 from the first edge 5 to the second edge 6 of the radiation protection drape, such as from one long side of the drape towards the opposing long side of the drape. Hence, the first section 3 may be partitioned from the second section 4 all the way from the first edge 5 to the second edge 6 of the radiation protection drape. Thus, the first section 3 may be separable from the second section 6. This provides for quickly at least partially removing the second section 4 from the patient, for example in an emergency situation, which enhances the patient safety of the radiation protection system 1.

In some embodiments, the first section 3 is partitioned from the second section 4 from the first edge 5 partly to the second edge 6. Thus the first section 3 may be only partly separated from the second section 4. Still the second section 4 may be folded along the separation for quick and easy access to the portion of the patient covered by the second section 4. At the same time, the risk of the second section 4 falling on the floor and being contaminated when folded to uncover the patient is reduced. This also enhances the patient safety.

In some embodiments, the first section 3 has a varying radiation protection level across the first section. For example, a section closer to the torso has a higher radiation protective level than a section towards the feet. In some embodiments, a higher radiation protective level is in the range of 0.35-0.7 mm lead equivalence, and a lower radiation protection is in the range of 0.25-0.5 mm lead equivalence. The radiation protection level may vary in steps or continuously from a first end of the first section 3 to an opposing end of the first section 4.

In some embodiments, such as is illustrated in FIGS. 3a-3b, the second section 4 may comprise a first portion 4a and a second portion 4b. The first portion 4a and the second portion 4b may be separated along an edge 7a, 7b of each of the first portion 4a and the second portion 4b. Thus, the first portion 4a and the second portion 4a can be separated from an unfolded position, such as illustrated in FIG. 3a, and be placed in a folded position, as is illustrated in FIG. 3b. Each or both the first portion 4a and the second portion 4b may be foldable, such as to a lateral side of the patient. Hence, quick and easy access to the area of the patient covered by the second section 4 is facilitated to enhance the patient safety. Also additional access to the radiated area of the patient at an area otherwise covered by any of the portions is facilitated while as much as possible of the patient is covered by the radiation protection drape 2, wherein radiation scatter is minimized. Hence, the second section 4 may be separated from upper end of the second section 4 towards an opposing lower end of the second section 4.

In some embodiments, each of the first portion 4a and the second portion 4b may comprise at least one fastener device 8a, 8b, 8c to temporarily fasten and unfasten the first portion 4a to/from second portion 4b. The fastener device 8a, 8b, 8c, may comprise a hook and loop system or magnets attached to each of the first portion 4a and the second portion 4b. Hence, accidental separation of the first portion 4a and the second portion 4b is provided. Thus accidental exposure to radiation scatter is avoided.

The first portion 4a and the second portion 4b may be sized to be overlapping in an area where they are separated. The overlap may be in the range of 1-10 cm. Such overlap provides for prevention of scatter radiation at the separation between the first portion 4a and the second portion 4b.

In some embodiments, the first portion 4a and the second portion 4b of the second section 4 are partially permanently attached to the first section 3 at the lower end each of each of the first portion 4a and the second portion 4b. Again, this prevents accidental separation of the first portion 4a and the second portion 4b when in the folded position and that any of the first portion 4a and the second portion 4b falls on the fall and becomes contaminated during an interventional procedure.

In some embodiments, a closure indicator is positioned at an overlap between the first section 3 and the second section 4, and/or between the first portion 4a and the second portion 4b. The closure indicator is configured to detect if the sections 3, 4 and/or the portions 4a, 4b are completely closed. Such a detection system may be provided by an electrical system with conductors that create a closed circuit when the sections 3, 4 and/or the positions 4a, 4b are completely closed. The electrical system may alternatively or additionally be wireless, such as a conductive system, for example including the magnets described herein. A closure indicator may also be provided for a table mounted radiation protection drape section 15 (described below), that detects that the drape is completely attached to the operating table. The closure indicator for the table mounted radiation protection drape section 15 may be provided separately from the closure indicator of any of the sections 3, 4 and/or the portions 4a, 4b. Alternatively, a common closure indicator is provided for the table mounted radiation protection drape section 15 and one or more of the sections 3, 4 and/or the portions 4a, 4b. The closure indicator can be provided together with any of the embodiments described herein. The closure indicator further enhances the safety of embodiments of the invention to ensure that no stray scatter passes the radiation barrier at a non-completely closed section of the radiation shielding protection system.

In some embodiments, such as is illustrated in FIG. 3b, the first portion 4a and the second portion 4b of the second section 4 are permanently or temporarily connected with one or several straps 9a, 9b, which have a width that is smaller than the length of the first portion 4a and the second portion 4b. This prevents accidental separation of the first portion 4a and the second portion 4b when in the folded position and that any of the first portion 4a and the second portion 4b falls on the fall and becomes contaminated during an interventional procedure. This also contributes to the safety of the embodiments of the invention.

The radiation protection drape 2 may comprise at least one fenestration 10 for a femoralis area. The fenestration 10 may be located along an edge of the first section 3 and/or an edge of the second section 4, along which the first section 3 and the second section 4 are partitioned. In the embodiments of the FIGS. 1, 3a, and 4, the fenestration 10 is a cut-out section at the end of the second section 4 located towards the first section 3. This provides for easy adjustment of the size and position of the fenestration 10 thus to gain optimal access to the entry point and minimize the open fenestration surface to reduce radiation leakage. In other embodiments, the fenestration 10 is a cut-out section at the end of the first section 3 located towards the second section 4. The cut-out section may have a square shape.

As is illustrated in FIG. 4, the second edge 6a of the radiation protection drape 2 may be located at the fenestration 10. Hence, the first section 3 and the second section 4 may be partitioned from the first edge 5 to the second edge 6a of the radiation protection drape 2. Hence, a portion of the first section 3 may be permanently joined to a portion of the second section 4. Furthermore, a first part of the fenestration 10 may be located in the first portion 4a and a second part of the fenestration 10 may located in the second portion 4b of the second section.

Any of the first section 3 and the second section 4 may be positioned under a sterile barrier. Hence, the radiation protection drape may be for multiple-use. This provides for having enhanced radiation protection properties of the radiation protection drape 2. Radiation protection drapes made for single uses less efficient radiation protective material for costs reasons, whereas multiple use drapes can be made of more efficient radiation protective materials. However, over time the multiple use drapes are more cost efficient. Hence, embodiments of the invention are cost efficient while at the same time providing enhanced safety.

As is illustrated in FIG. 1, the radiation protection drape may comprise a third section 11 separated from the first section and the second section. The third section 11 may be sized to partially cover the fenestration 10 of the radiation protection drape 2. The third section may have a length in the range of 20-60 cm and a width in the range of 40-80 cm. Hence, the fenestration 10 can be sufficiently large to access both left and right femoralis area of the patient. At the same time, any of the left and right femoralis area of the patient may be covered by the third section 11. This reduces scatter from any unprotected area, wherein the safety is enhanced while at the same time enhancing the flexibility of the radiation protection system 1.

In some embodiments, such as is illustrated in FIG. 2 by the second section 3, the radiation protection drape 2 may be sized to extend over the rim 12 of an operating table 13 when positioned over a patient lying on the operating table 13. Furthermore, the radiation protection drape 2 may be sized to extend over the rim 12 of the operating table 13 and at least partially towards to the floor on at least one side of the operating table 13. In some embodiments, the radiation protection drape 2 extends at least 10 cm over the rim of the operating table 13 when laying over a patient. Hence, the radiation protection drape may cover the side of the patient when lying on the operating table. This provides enhanced protection from radiation scatter and enhanced safety.

As is illustrated in FIGS. 1a and 1, the radiation protection shielding system 1 may further comprise at least one floor or ceiling mounted radiation shielding screen 14 to be positioned between clinical staff and a radiated area at an upper body portion of the patient. This reduces radiation scatter towards staff from any unprotected radiation area of the patient, such as from the upper part of the torso of the patient.

As is illustrated in FIG. 4, the radiation protection shielding system 1 may further comprise at least one floor or table mounted radiation protection drape section 15 configured to cover at least a part of an area under the operating table 13. The floor or table mounted radiation protection drape section 15 may be being sized to cover least one long side and/or at least one foot end side of the operating table 13, such as is illustrated in FIG. 4.

As is illustrated in FIG. 2, the radiation protection drape, such as any of the first section 3 and the second section 4, may comprise at least one recess 16 along an edge of the drape. The recess may be sized and positioned to prevent that the drape 2 covers any control panel of medical equipment.

The radiation protection drape 2 and any section of the radiation protection system may have a radiation protection level corresponding to 0.1 to 1 mm lead equivalency, most preferably 0.35-0.7 mm lead equivalency.

As illustrated in FIG. 1a, the radiation protection shielding system may comprise a radiation protection garment 17. The radiation protection garment 17 may comprise at least one panel sized to extend over at least the front side of a torso of a wearer. The panel may have a radiation protection level of 0.05 to 0.35 mm lead equivalency. Hence, the radiation protection drape 2 may have a higher radiation protective level than the panel of the radiation protection garment 17. Furthermore, when made of the same material, the panel of the radiation protection garment 17 may be lighter per surface area than the radiation protection drape 2. The staff may be protected from all or substantially all of the radiation scatter from the patient by the radiation protection drape 2, and from any residual radiation scatter by the radiation protection garment 17. Since the residual radiation scatter will be low, the radiation protection garment 17 can be made lighter. The radiation protection garment 17 may e.g. be an apron with front protection and/or back protection. Other embodiments of garments comprise a set of shirt and pants, with front and/or back protection. The radiation protective material in the radiation protection garment 17 may optionally comprise a textile radiation protective material including radiation protective filaments positioned as a weave. Thus the embodiments of the invention improves working environment for the staff in the operating room, while at the same time reduce the risk for work related orthopedic injuries and disorders to the back, neck, shoulders, hips and knees as well as chronic fatigue as a consequence of carrying heavy personal lead protection. These benefits are provided while at the same time improving the radiation protection for the staff.

The radiation protection drape 2 and/or garment 17 of the embodiments of the invention may be made of the material disclosed in WO2014163574 by the same applicant as the present invention and which is incorporated herein in its entirety for all purposed. For example, the radiation protection drape 2 and/or garment 17 of the embodiments may comprise one or several layers of the material, as is disclosed in WO2014163574, such as to provide a varying radiation protection, as discussed above.

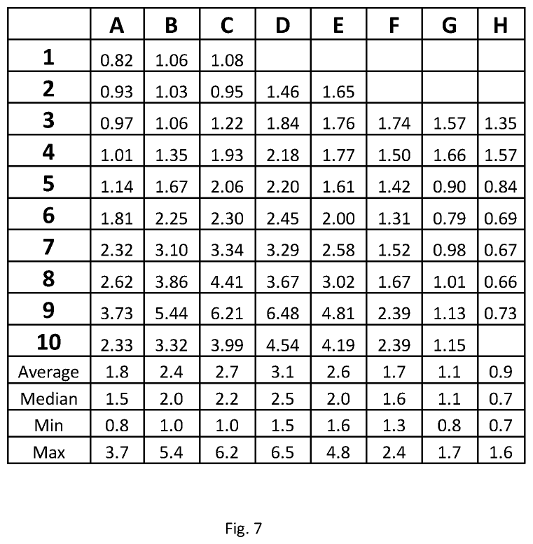

In FIGS. 5-7, data from three examples are shown. Example 1 and Example 2 includes data from embodiments of the invention wherein a radiation protection drape was laid over the patient area on the operating table during measurements. Example 3 is comparative data from a test wherein radiation protection laid over the patient area on the operating table to protect from radiated scatter was not used. The letters A-H and numbers 1-10 in the tables of FIGS. 5-7 constitute positions in a matrix where A is positioned on the front right chest of the first operator whereas the position H is positioned underneath the left arm of said operator. The position 1 is on the upper chest of the first operator and the position 10 is on the knee area of said operator. The unit in the tables of FIGS. 5-7 is milliSievert (mSv) and is extrapolated to 250 procedures to represent an estimated annual dose to the first operator.

### Example 1

The table in FIG. 5 includes data from example 1. A radiation protection shielding system according to the embodiments of the invention was made by utilizing a radiation protection drape of 0.5 mm lead equivalence covering the patient from the chest down to cover the feet with a fenestration in the femoralis area, a table mounted lead shield (Rampart M1128) mounted between the patient head and the operator having a lead equivalence of 1.0 mm, and table mounted radiation protection curtain having a lead equivalence of 0.5 mm along the side of the operating table thus to protect from radiation coming underneath the table. The radiation protecting drapes were overlapping the radiation protecting table curtains thus to avoid stray radiation to pass the radiation protection barrier. In the study, 34 interventions were conducted (Coronary angiography, PCI, CTO-PCI, alcohol septal ablation and aortic valve intervention). A radiation protection apron with a lead equivalence of 0.5 mm had attached 72 thermoluminescent dosimeters (DXT-RAD RingletsTLD-100), positioned over the outside front of the apron on torso down to the knee area of the first operator. During each intervention, the data was registered and analysed at the end of the study. The results were extrapolated to 250 procedures to represent an estimated annual dose to the first operator. The mean personal dose equivalent, extrapolated to annual dose was measured to 0.3 mSv outside the radiation protection apron of the first operator. The table of FIG. 5 shows the measured dose at any position on the first operator's abdomen.

### Example 2

The table in FIG. 6 includes data from example 2. A radiation protection system according to the embodiments of the invention was made by utilizing a radiation protection drape of 0.5 mm lead equivalence covering the patient from the chest down to cover the feet with a fenestration in the femoralis area, a roof mounted lead screen, having a lead equivalence of 0.5 mm, (Mavig GmBH) placed between the patient head and the operator, and table mounted radiation protection curtain having a lead equivalence of 0.5 mm along the side of the operating table thus to protect from radiation coming underneath the table. The radiation protecting drapes were overlapping the radiation protecting table curtains thus to avoid stray radiation to pass the radiation protection barrier. In the study, 38 interventions were conducted (Coronary angiography, PCI, CTO-PCI and aortic valve intervention). A radiation protection apron with a lead equivalence of 0.5 mm had attached 72 thermoluminescent dosimeters (DXT-RAD RingletsTLD-100), positioned over the outside front of the apron on torso down to the knee area of the first operator. During each intervention, the data was registered and analysed at the end of the study. The results were extrapolated to 250 procedures to represent an estimated annual dose to the first operator. The mean personal dose equivalent, extrapolated to annual dose was measured to 0.3 mSv outside the radiation protection apron of the first operator. The table of FIG. 6 shows the measured dose at any position on the first operator's abdomen.

### Example 3

The table in FIG. 7 includes data from example 3. A conventional system was used where there were no radiation protection over the patient, a roof mounted lead screen, having a lead equivalence of 0.5 mm, (Mavig GmBH) placed between the patient and the staff using a lead radiation protection apron having a lead equivalence of 0.5 mm (Mavig GmBH). A table mounted radiation protection curtain having a lead equivalence of 0.5 mm along the side of the operating table was used to protect from radiation coming underneath the table. In the study, 35 interventions were conducted (Coronary angiography, PCI, CTO-PCI and aortic valve intervention). A radiation protection apron had attached 72 thermoluminescent dosimeters (DXT-RAD RingletsTLD-100), positioned over the front side of the apron on torso down to the knee area of the first operator. During each intervention, the data was registered and analysed at the end of the study. The results were extrapolated to 250 procedures to represent an estimated annual dose to the first operator. The mean personal dose equivalent, extrapolated to annual dose was measured to 2.1 mSv outside the radiation protection apron of the first operator. The table of FIG. 7 shows the measured dose at any position on the first operator's abdomen.

### Conclusions from Example 1, Example 2, and Example 3

Using embodiments of the invention as visualized by Examples 1 and 2 reduces the exposure of the first operator with a factor 7, based on mean values, as compared to a conventional radiation protection set-up as displayed in Example 3. In individual positions over the body of the first operator, such as on the left chest side of the first operator (position C-D) in the lower body (position 5-9), the reduction may be greater.

The lead equivalency as described herein may be measured according to the standard IEC 61331-1:2014.

### Example 4

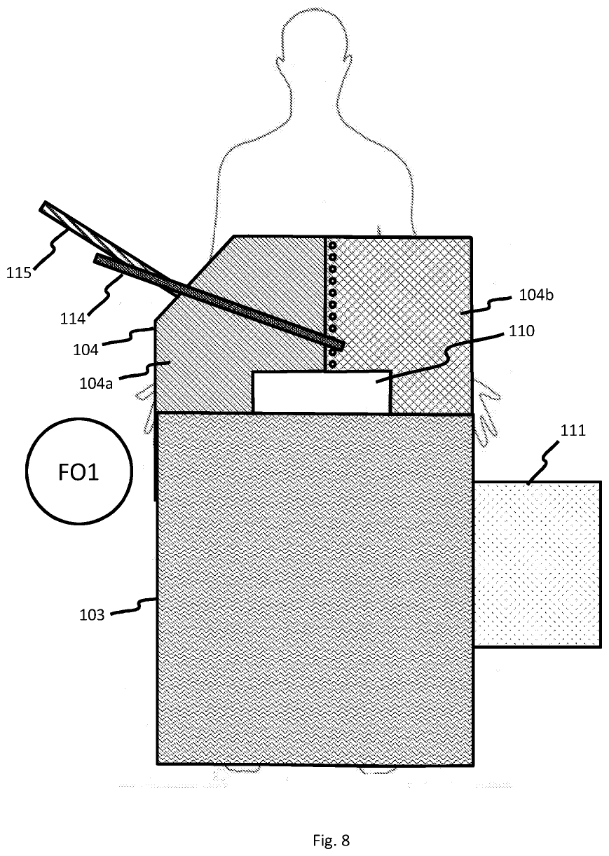

Measuring radiation exposure illustrates the benefits of radiation protection for the health care worker using embodiments of the invention. The presented data in Table 1 below have been collected from cardiology interventions within the specialty of Percutaneous coronary intervention, PCI. Dosimeters have been placed on the first operator, who was positioned next to the operating table in the area around the radialis position and towards the leg of the patient. In FIG. 8, the position relative to the operating table of the first operator is indicated with FO1.

The X-ray equipment used during the measurements was Philips Multi Diagnost Eleva FD (Koninklijke Philips Electronics N.V.) The operating table was at zero-degree angle and an under-table X-ray tube position was used with normal rotation angles for cardiovascular interventions. The tube voltage was at approximately 90 kV. In all measurements, RaySafes (Unfors RaySafe AB, Billdal, Sweden) i3 Real-time dosimetry System was used to indicate the amount of scattered radiation exposed to the first operator. The dosimeters were placed at four different dosimeter positions, which were all on the outside of the radiation protection apron of the first operator abdomen. In table 1, the four different dosimeter positions (DP) on the first operator are indicated as DP1, DP2, DP3, and DP4. DP1 was positioned at the right side of the chest, DP2 was at the left side of the chest, DP3 was on the left side of the hip, and PD4 was placed center belly. During the interventions, a ceiling mounted lead screen 114 from Mavig (Mavig Stahlgruberring 5, Munich, Germany) of 0.5 mm Lead equivalence was used. A radiation protection drape section 115 formed a table skirt on the side of the operating table from the foot end to the shoulder level of the patient. The table skirt was from Kenex (Kenex, Greenway, Harlow Business park, Harlow, England) of 0.5 mm Lead equivalence was placed on the side of the operating table together with table skirt extension mounted in a hinge from the operating table (partially illustrated in FIG. 8).

FIG. 8 illustrates a radiation protection shielding system according to embodiments of the invention as described above was used during the measurements of example 4. The radiation protection shielding system comprised a radiation protection drape that was sized to partially cover the patient from a proximity of the radiated area of the patient to the feet of the patient. The feet were covered by the radiation protection drape. The radiation protection drape comprised at least a first section and a second section. The first section was partitioned from the second section from a first edge of the radiation protection drape towards a second edge of the radiation protection drape.

The first section was partitioned from the second section from the first edge to the second edge of the radiation protection drape and being separable from the second section. However, this is not necessary in all embodiments of the invention.

The first section was partitioned from the second section from the first edge partly to the second edge, and the first section was partly separated from the second section. However, this is not necessary in all embodiments of the invention.

The first section had a varying radiation protection level across the first section. However, this is not necessary in all embodiments of the invention.

The second section comprised a first portion and a second portion, where the first portion and the second portion were separated along an edge of each of the first portion and the second portion, and comprised a fastener to temporarily fasten and unfasten the first portion to/from second portion. However, this is not necessary in all embodiments of the invention.

The radiation protection drape comprised one fenestration for a femoralis area of the patient, wherein the fenestration was located along the partition of the first section and the second section. However, this is not necessary in all embodiments of the invention.

The second edge was located at the fenestration, the first section and the second section were partitioned from the first edge to the second edge. However, this is not necessary in all embodiments of the invention.

The radiation protection drape comprised a third section separated from the first section and the second section and was sized to partially cover the fenestrated area of the radiation protection drape. However, this is not necessary in all embodiments of the invention.

The radiation protection shielding system comprised a fastening device, in the form of magnets located at where the first portion was partitioned from the second portion. However, this is not necessary in all embodiments of the invention.

The radiation protection drape was sized to extend over the rim of the long side of the operating table facing the first operator, when positioned over the patient lying on the operating table, and towards to the floor on the side of the operating table, about 10 cm over the rim of the operating table, such as to cover the side of the patient when laying on the operating table. However, this is not necessary in all embodiments of the invention.

The ceiling mounted radiation shielding screen 114 was positioned between clinical staff and the radiated area at an upper body portion of the patient. However, this is not necessary in all embodiments of the invention.

The radiation protection shielding system comprised a table mounted radiation protection drape section that was configured to cover at least a part of the area under the table.

In combination with the radiation protection drape, a radiation protection garment in the form of an apron was used by the first operator. However, this is not necessary in all embodiments of the invention.

On the patient, the radiation protection drape with a first section 103 and a second section 104 had a 0.5 mm Lead equivalence and was mounted as illustrated in FIG. 8. The second section 104 formed a chest blanket), and was placed on the lower abdomen of the patient. The second section 104 comprised a woven radiation protection material from the applicant of the present invention, Texray AB (Texray AB, Stena Center, Göteborg, Sweden), further described in EP EP2981975B1 (Radiation Protective Material), which is incorporated herein for all purposes. The woven radiation protection material of the second section 104 comprised 0.50 mm Lead equivalence. Two layers of the woven radiation protection material, each of 0.25 mm Lead equivalence, were placed on top of each other to increase the protection level to approximately 0.50 mm Lead equivalence and formed the second section 104. In the embodiments of example 4, the second section was partitioned along the center of the second section to form the first portion 104a and the second portion 104b as described above with regard to various embodiments. The first portion 104a and the second portion 104b, were overlapping and interconnected by magnets. This contributed to firmly hold the first portion 104a and the second portion 104b together and also allowed for easy access to the patient in the case of emergency procedures, as also displayed in FIGS. 3 and 4. The first section 103 formed a Foot Blanket that was placed on the patient from the femoralis area and covered the lower part of the patient body, including the feet. The first section 103 comprised the same woven radiation protection material as the second section 104. The uppermost 20 cm (towards the femoralis area) of the first section 104 comprised double layers of the woven radiation protection material, each layer comprising 0.25 mm Lead equivalence. The lower part of the first section 103 (towards the feet) comprised one layer of the woven radiation protection material with 0.25 mm Lead equivalence. To cover the fenestration 110 in the femoralis, a third section 111 of the radiation protection system that formed a patch was used (positioned to the side of the operating table in FIG. 8 for illustration purposes). The third section 111 had limited effect for the reduction of radiation but was used in this specific example to mitigate any risk of scatter radiation originating from the femoralis area. The third section 111, comprised the same woven radiation protection material as the second section 104. Two layers of 0.25 mm Lead equivalence each was used for the third section 111. During the interventions, the third section 111 was positioned over the fenestration (Z4).

In Table 1, the radiation exposure to the dosimeters, placed on the first operator in the intervention field when the embodiments of the Invention as described in example 4, is presented. The values in Table 1 are in micro Sievert, and the average exposure is the average registered exposure towards the First operator. The average reduction efficacy denotes the quotient between the average exposure to the first operator and the emitted dose, represented by a dosimeter placed under the head of the patient. This value may vary pending on the type of intervention and the time of image guiding needed during the intervention. As can be seen in Table 1, the radiation exposure registered under the head of the patient was highest for Patient 4, which is an indication that image guiding with radiation was used more than for Patients 1 to Patient 3. For Patient 2, the radiation exposure was the lowest.

It can be concluded that the embodiment in Example 4 reduces the radiation exposure towards the first operator effectively, with a 99% to 100% reduction of radiation. With the registered level of radiation emitted to the first operator, the need for body worn radiation protection is reduced and a radiation protection apparel of lower protection level can be carried, or no body worn radiation protection apparel is needed. Hence, embodiments of the invention, as illustrated with example 4, provide numerous benefits for clinical staff and patient as described above in the sections “Summary of the Invention” and “Description of Embodiments”.

TABLE 1
Radiation Exposure (microSievert)

| | Patient | Patient | Patient | Patient |
|---|---|---|---|---|
| Dosimeter position | 1 | 2 | 3 | 4 |
| Under head of patient | 65.4 | 22.3 | 65.1 | 272.2 |
| First operator DP1 (upper chest, right) | 0.0 | 0.3 | 0.0 | 0.1 |
| First operator DP2 (upper chest, left) | 0.0 | 0.3 | 0.0 | 0.4 |
| First operator DP3 (hip, left) | 0.3 | 0.3 | 0.0 | 0.5 |
| First operator DP4 (belly, middle) | — | — | 0.5 | 3.2 |
| Average radiation exposure (first | 0.1 | 0.3 | 0.1 | 1.1 |
| operator) | | | | |
| Average reduction efficacy (%) | 100% | 99% | 100% | 100% |

As will be apparent, the features and attributes of the specific embodiments disclosed above may be combined in different ways to form additional embodiments, all of which fall within the scope of the present disclosure.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

Any process descriptions, elements, or blocks in the flow diagrams described herein and/or depicted in the attached figures should be understood as potentially representing modules or segments. Alternate implementations are included within the scope of the embodiments described herein in which elements or functions may be deleted, executed out of order from that shown or discussed, including substantially concurrently or in reverse order, depending on the functionality involved, as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure and protected by the following claims.

The present invention has been described above with reference to specific embodiments. However, other embodiments than the above described are equally possible within the scope of the invention. Different method steps than those described above may be provided within the scope of the invention. The different features and steps of the invention may be combined in other combinations than those described. The scope of the invention is only limited by the appended patent claims.

1. A radiation protection shielding system for a patient lying on an operating table, the system comprising:
a radiation protection drape sized to at least partially cover the patient from a proximity of a radiated area of the patient to the feet of the patient, wherein the radiation protection drape comprises at least a first section and a second section, wherein the first section is partitioned from the second section along a partition between the first section and the second section that extends at least partially from a first edge of the radiation protection drape towards a second edge of the radiation protection drape.

2. The radiation protection shielding system according to claim 1, wherein the partition extends from the first edge to the second edge of the radiation protection drape, the first section being separable from the second section along the partition.

3. The radiation protection shielding system according to claim 1, wherein the partition extends from the first edge partly to the second edge, the first section being partly separable from the second section along the partition.

4. The radiation protection shielding system according to claim 1, wherein the first section has a varying radiation protection level across the first section.

5. The radiation protection shielding system according to claim 1, wherein the second section comprises a first portion and a second portion, wherein the first portion and the second portion are separably fastened to each other along an edge of each of the first portion and the second portion.

6. The radiation protection shielding system according to claim 1, wherein the radiation protection drape comprises a fenestration for a femoralis area of the patient, wherein the fenestration is located along the partition between the first section and the second section.

7. The radiation protection shielding system of claim 6, wherein the second edge is located at the fenestration, and wherein the partition between the first section and the second section extends from the first edge to the second edge.

8. The radiation protection shielding system according to claim 1, wherein the radiation protection drape includes a fenestrated area, and wherein the radiation protection drape comprises a third section configured to partially cover the fenestrated area of the radiation protection drape.

9. The radiation protection shielding system according to claim 1, further comprising a fastening device located at the partition between the first section and the second section.

10. The radiation protection shielding system according to claim 1, wherein the operating table has a rim, and wherein the radiation protection drape is sized to extend over the rim of the operating table, when positioned over the patient lying on the operating table, and towards a floor on at least one side of the operating table.

11. The radiation protection shielding system according to claim 1, further comprising a radiation shielding screen configured to be positioned between clinical staff in the proximity of the operating table and a radiated area at an upper body portion of the patient.

12. The radiation protection shielding system according to claim 1, wherein the radiation protection drape further comprises a third section configured to cover an area under the operating table.

13. The radiation protection shielding system according to claim 1, wherein the radiation protection drape has a radiation protection level corresponding to 0.1 to 1 mm lead equivalency.

14. The radiation protection shielding system according to claim 1, further comprising a radiation protection garment.

15. The radiation protection shielding system according to claim 14, wherein the radiation protection garment comprises a panel sized to extend over at least a torso front side of a wearer, the panel having a radiation protection level of 0.05 to 0.35 mm lead equivalency.

16. (canceled)

17. The radiation protection shielding system according to claim 1, further comprising a closure indicator positioned at an overlap between at least the first section and the second section.

18. A method for covering a patient with a radiation protection shielding system during a medical intervention including radiation with ionizing radiation, comprising:
at least partially covering the patient with a radiation protection drape from a proximity of a radiated area of the patient to the feet of the patient, wherein said covering comprises covering a first portion of the patient with a first section of the radiation protection drape and a second portion of the patient with a second section of the radiation protection drape, wherein the first section is partitioned from the second section from a first edge of the radiation protection drape towards a second edge of the radiation protection drape.

19. The radiation protection shielding system according to claim 1, wherein the radiation protection drape includes antimony as a radiation attenuating ingredient.

20. The radiation protection shielding system of claim 14, wherein the radiation protection drape includes a first radiation attenuating ingredient, and the radiation protection garment includes a second radiation attenuating ingredient different from the first radiation attenuating ingredient.

21. The radiation protection shielding system of claim 20, wherein the first radiation attenuating ingredient comprises antimony, and the second radiation attenuating ingredient comprises at least one of bismuth and wolfram.
